(19) United States
(12) Patent Application Publication
(10) Pub. No.: US 2017/0367872 A1
(43) Pub. Date: Dec. 28, 2017

(54) WASTE COLLECTION BAND

(71) Applicant: Angela M. Cantrell, Dallas, TX (US)

(72) Inventor: Angela M. Cantrell, Dallas, TX (US)

(21) Appl. No.: 15/633,455

(22) Filed: Jun. 26, 2017

Related U.S. Application Data
(60) Provisional application No. 62/355,186, filed on Jun. 27, 2016.

(51) Int. Cl. A61F 5/449; A61F 5/44

(52) U.S. Cl. CPC A61F 5/449; A61F 5/4401

(57) ABSTRACT

A stoma band for collecting waste or other fluids from a stoma includes a belt portion with a first side that faces away from a user wearing the stoma band, a second side that faces toward the user wearing the stoma band, a first end, and a second end. The stoma band also includes a first fastener on the first end of the belt portion and a second fastener on the second end of the belt portion. The stoma band also includes a collection device adapted to be positioned between the second side of the belt portion and the user so that the collection device is proximal to a stoma of the user.

### BACKGROUND OF THE INVENTION

Complications arising from various health conditions sometimes result in the need for a patient to have one or more stomas that provide an artificial opening from outside the body into a cavity within the body. Common stomas include ileostomies, colostomies, and urostomies. These stomas are used to evacuate wastes from the body. As used herein, waste is intended to generally refer to any substance desired to be removed from the body, such as, for example, urine and fecal matter. While stomas are very beneficial and necessary to the patient, living with a stoma presents certain difficulties to the patient's day-to-day life. For example, waste that is evacuated from the stoma must be collected and removed from the patient. Various methods have been developed over the years to help patients manage stoma waste collection and removal. However, the collection and removal process often results in skin irritation, discomfort, and limited mobility. Each of these physical issues can also result in emotional distress, especially when the patient is a child.

### SUMMARY

The present invention relates to a system for waste collection and removal that provides the user with improved comfort and mobility compared to prior systems. In a typical embodiment, the waste collection and removal system includes a band that is adapted to wrap around a portion of the user's body where the stoma is located and a collection device that is adapted to collect waste as it is evacuated from the user. In some embodiments, the band may include various cutouts or contours that conform to the user's body to provide a comfortable fit. In some embodiments, the band may include one or more straps that interact with the user's body to help securely position the band on the user's body. The collection device is adapted to fit between the band and the user's body where the stoma is located. In some embodiments, the band is adapted to receive additional collection devices for each stoma that the patient has. In a typical embodiment, the collection device is a pad that absorbs waste as it is evacuated out of the stoma. The band is adapted to be easily removed from and secured to the user to facilitate changing out a used collection device for a new one.

In some embodiments, a stoma band includes a belt portion having a first side that faces away from a user wearing the stoma band, a second side that faces toward the user wearing the stoma band, a first end, and a second end. The stoma band also includes a first fastener on the first end of the belt portion and a second fastener on the second end of the belt portion. The stoma band also includes a collection device adapted to be positioned between the second side of the belt portion and the user so that the collection device is proximal to a stoma of the user.

In some embodiments, the stoma band further includes a second collection device that is adapted to be positioned between the second side of the belt portion and the user so that the second collection device is proximal to a second stoma of the user.

In some embodiments, the collection device is removably secured to the second side of the belt portion by at least one of a hook and loop fastener, a button, and a snap. In some embodiments, the collection device is made of a material comprising at least one of bamboo, cotton, hemp, modal fabrics, polyester, fleece, wool, and polyurethane laminate.

In some embodiments the first fastener includes at least one removable fastener that can be removed to adjust a length of the belt portion.

In some embodiments, the stoma band further includes a cut-line disposed between adjacent removable fasteners.

In some embodiments, the stoma band includes a contoured portion disposed along a length of the belt portion. In some embodiments, the stoma band includes a second contoured portion disposed along the length of the belt portion.

In some embodiments, the second end comprises a tapered end.

In some embodiments, the first fastener is disposed on the first side of the belt portion and the second fastener is disposed on a second side of the belt portion.

In some embodiments, the stoma band includes a shoulder strap attached to the belt portion. In some embodiments, the stoma band includes a leg strap attached to the belt portion.

In some embodiments, the stoma band is attached to at least one of a shirt, pants, shorts, and an undergarment. In some embodiments, the stoma band is integrated into an article of clothing.

In some embodiments, the first and second fasteners are hook and loop fasteners. In some embodiments, the first and second fasteners are button snaps. In some embodiments, the first and second fasteners are buckles.

In some embodiments, the stoma band includes a pouch disposed on the belt portion.

In some embodiments, the pouch is disposed on the second side of the belt portion.

In some embodiments, the collection device extends across a waste of the user.

### BRIEF DESCRIPTION OF THE DRAWINGS

A more complete understanding of embodiments of the present invention may be obtained by reference to the following Detailed Description when taken in conjunction with the accompanying Drawings wherein:

FIG. 1 is front view of a stoma band;

FIG. 2 is a front view of a contoured stoma band;

FIG. 3 is a front view of a stoma band with a customizable length;

FIG. 4 is partial front view of a stoma band with a tapered end;

FIG. 5 is a front view of a stoma band with a storage pouch;

FIG. 6 is an isometric view of a contoured stoma band that includes supportive straps; and

FIG. 7 is an illustration of a stoma band system secured around a user.

### DESCRIPTION

Some medical conditions result in a patient requiring one or more stomas. A stoma is a passageway that is formed through a patient's skin to allow for communication of a substance out of or into the patient's body. For example, a stoma may be necessary to remove fecal matter from the small intestine (ileostomy), large intestine (colostomy), or to remove urine from the bladder (urostomy). As waste is evacuated from the patient, it must be collected and eventually removed from the patient. To date, wastes collected from ileostomies, colostomies, and urostomies are often collected in bags that are secured to the patient. A typical bag setup includes a tube that is secured to the patient. This is often accomplished by using an adhesive that semi-permanently secures the tube to a surface of the patient's skin that surrounds the stoma. The securement is semi-permanent in that it can lasts for days, weeks, or even months depending on the particular application. However, the adhesive eventually weakens and comes free from the patient's skin. A common side of effect of securing the tube or with an adhesive is irritation to the patient's skin, which can sometimes be severe. Before a tube can be re-secured to the patient's stoma, the patient's skin must be allowed to heal. During the healing process, it is difficult for the patient to have a bag secured to their stoma to collect evacuated waste. This has been generally undesirable as it makes collection and removal of waste more difficult.

The waste collection system of the present application allows for the collection of evacuated waste without requiring a tube to be adhered to the patient's skin. By not requiring a tube, the waste collection system of the present application provides a more comfortable patient experience and also provides a more discrete solution that can be more easily concealed beneath the patient's clothing. Concealing the waste collection system can also increase the patient's emotional wellbeing because the concealed system draws less attention from others. The lack of a tube also allows the patient to have increased movement and flexibility without worry of weakening securement of a tube placed in the stoma.

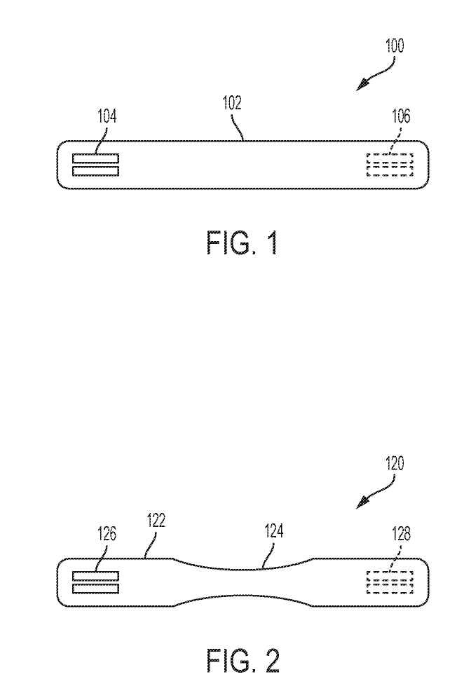

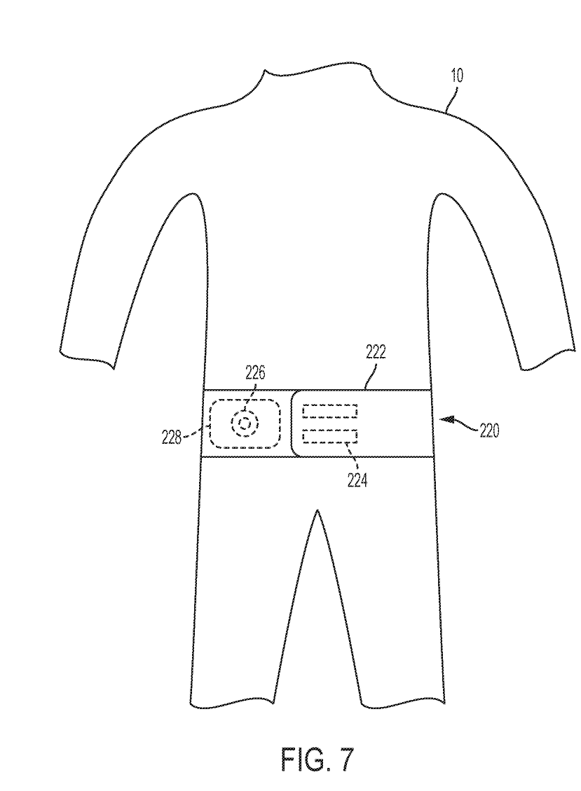

Referring now to FIG. 1, a front view of a stoma band 100 is shown. The stoma band 100 includes a belt portion 102 and a fastener 104 and a fastener 106. In a typical embodiment, the stoma band 100 is adapted to be wrapped around a user's waste or mid-section. When in use, the fasteners 104 and 106 are joined together so that the stoma band 100 forms a loop that is worn like a belt. In a typical embodiment, the fastener 104 is on a first side of the belt portion 102 and the fastener 106 is on a second side of the belt portion 102 (indicated as dashed lines) so that the belt portion 102 is fastened together by overlapping end portions of the belt portion 102. The first side of the belt portion 102 may be an outside of the belt portion 102 and the second side of the belt portion 102 may be an inside of the belt portion 102 (defined relative to the patient). The stoma band 100 is adapted to secure one or more collection devices over a stoma (best seen in FIG. 7). In a typical embodiment, the collection device is an absorptive pad. Various types of absorptive pads may be used. Absorptive pads made from materials that are highly absorptive, such as, for example, diapering-type materials, are suited for use as collection devices with various embodiments of the stoma band 100. For example, pads may be made by one or more of the following: bamboo, cotton, hemp, modal fabrics, polyester, fleece, wool, PUL (Polyurethane Laminate, e.g., a blend of 83% polyester, 15% polyurethane, and 2% agglutinant), and the like. In general, materials capable of absorbing large of amounts of liquid may be used as collection devices. In some embodiments, the absorptive nature of the collection device also helps reduce odors from becoming overtly noticeable. Also, materials that tend to wick moisture away from the skin may also be used as collection devices. Wicking moisture away from the skin of the user helps prevent skin irritation and allows the user to continuously wear the stoma band 100 for long periods of time without experiencing discomfort. In some embodiments, the absorptive pad includes an adhesive backing that adheres to the belt portion 102 to help limit movement of the absorptive pad relative to the belt portion 102. The collection device will be discussed in more detail below.

In some embodiments, the collection device provides some padding that covers the stoma. Stomas tend to be highly sensitive to touches, bumps, and contact, the result of which is moderate to severe discomfort and/or pain. The collection device of the present invention, in addition to absorbing evacuated waste materials, provides some padding that helps protect the stoma and reduces pain from incidental contact to the stoma.

A length and width of the belt portion 102 may be sized as desired to accommodate different users. In a typical embodiment, the belt portion 102 is made of a fabric material that is slightly stretchable. In a typical embodiment, the belt portion 102 is made of a fabric material that is breathable to help reduce skin irritation. In some embodiments, the belt portion 102 is made of a fabric material that tends to wick moisture away from the skin to help reduce skin irritation. In other embodiments, various fabrics or materials may be used, such as, for example, neoprene, rubbers, plastics, cottons, synthetic fabrics, and the like. The stoma band 100 may come in a variety of sizes, such as, for example, small, medium, and large. In a typical embodiment, the belt portion 102 is generally straight or linear. In other embodiments, the belt portion 102 may be curved or arcuate. Generally speaking, the stoma band 100 may be shaped as needed to ensure a close, snug fit of the stoma band 100 around the user's stoma.

In the embodiment shown in FIG. 1, fasteners 104 and 106 are hook and loop fasteners. Hook and loop fasteners allow some adjustability of the diameter of the stoma band 100 as the amount of overlap between the fasteners 104 and 106 may be controlled by the user. In other embodiments, fasteners 104 and 106 may be any of a variety of other fasteners. For example, the fasteners 104 and 106 may be button snaps, hooks, zippers, magnets, buckles (e.g., the fastener 104 comprises a portion of a traditional belt with holes and the fastener 106 comprises a portion of a traditional belt with a buckle), and the like. In other embodiments, the belt portion 102 may be a continuous piece that needs no fasteners. In such an embodiment, the user may slide the stoma band 100 into position by, for example, stepping into the band or pulling the band down over their head.

Referring now to FIG. 2, a front view of a contoured stoma band 120 is shown. The contoured stoma band 120 includes a belt portion 122 that includes a contoured portion 124, a fastener 126, and a fastener 128. The contoured stoma band 120 is similar to the stoma band 100, but the belt portion 122 includes the contoured portion 124. In the embodiment shown in FIG. 2, the contoured portion 124 is adapted to be positioned near a user's back. The contoured portion 124 allows better movement by the user and also increases user comfort. In other embodiments, a location and shape of the contoured portion 124 may be changed as desired to fit different body types or for use with other parts of a user's body. In a typical embodiment, the fasteners 126 and 128 are similar to the fasteners 104 and 106.

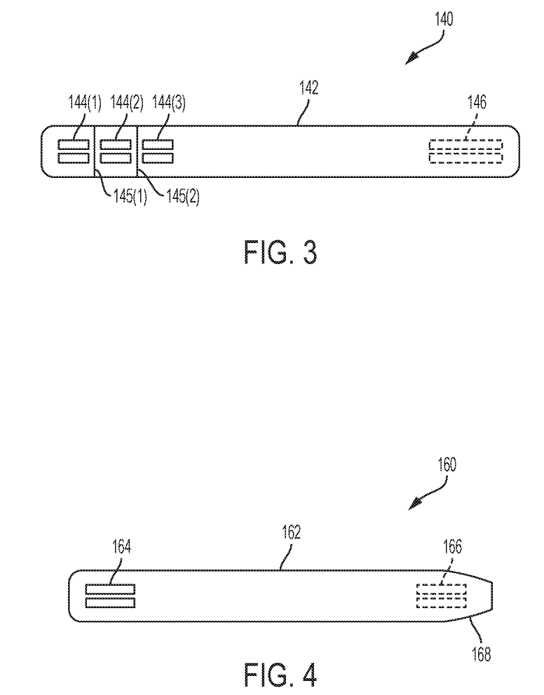

Referring now to FIG. 3, a front view of a customizable stoma band 140 is shown. The customizable stoma band 140 includes a belt portion 142, removable fasteners 144(1), 144(2), and 144(3), and a fastener 146. The customizable stoma band 140 is similar to the stoma band 100, but includes the removable fasteners 144(1), 144(2), and 144(3). The removable fasteners 144(1), 144(2), and 144(3) allow for a single off-the-shelf stoma band to be customizable to provide different lengths for a variety of different users. If a shorter-length stoma band 142 is needed, a user may remove sections of the belt portion 142 that includes the fasteners 144(1) and 144(2) by cutting along a cut-line 145(2) to shorten a length of the belt portion 142. If a medium-length stoma band is needed, a user may cut along a cut-line 145(1) to remove a section of the belt portion 142 that includes the fastener 144(1). If a large stoma band is needed, the user can use the customizable stoma band 140 without removing any of the fasteners 144(1), 144(2), and 144(3). The cut-lines 145(1) and 145(2) provide an indication as to where the belt portion 142 may be cut to remove a removable fastener 144(1) or 144(2). In some embodiments, the cut-lines 145(1) and 145(2) may comprise perforations that weaken the belt portion 142 to make removal of the removable fasteners 144(1) or 144(2) easier. In other embodiments, more or fewer removable fasteners 144 and cut-lines 145 may be added as desired to provide more or less customizability as desired.

Referring now to FIG. 4, a front view of a stoma band 160 with a tapered end 168 is shown. The stoma band 160 includes a belt portion 162, a fastener 164, a fastener 166, and the tapered end 168. The stoma band 160 is similar to the stoma band 100, but the belt portion 162 includes the tapered end 168. The tapered end 168 allows for some flexibility in aligning the tapered end 168 relative to an opposite end of the belt portion 162. For example, the tapered end 168 helps prevent an edge of the tapered end 168 from hanging over the opposite end of the belt portion 162, resulting in a more pleasing securement of the stoma band 160 around the user. In a typical embodiment, the fasteners 164 and 166 are similar to the fasteners 104 and 106.

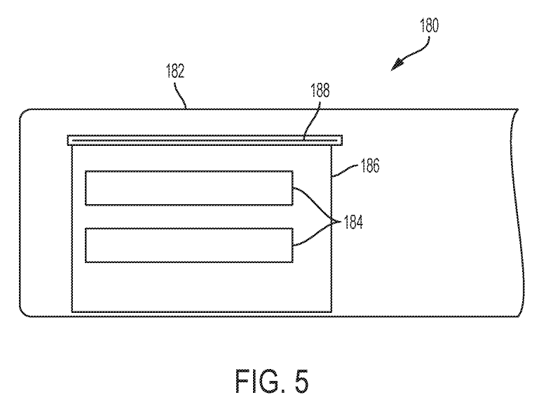

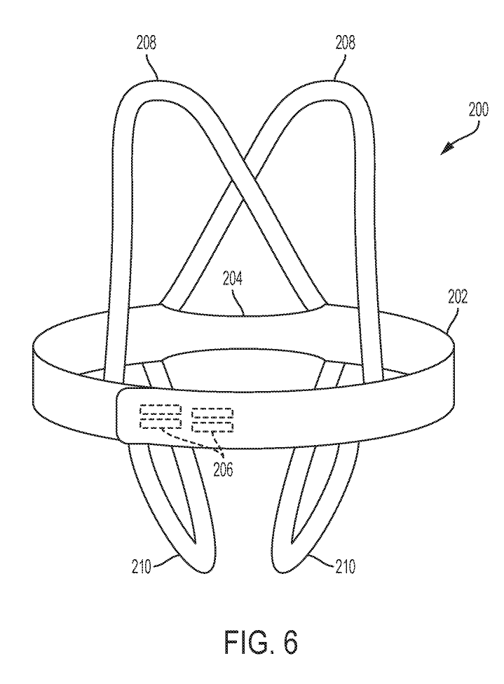

Referring now to FIG. 5, a partial front view of a stoma band 180 with a storage pouch 186 is shown. The stoma band 180 includes a belt portion 182, a fastener 184, and the storage pouch 186. The stoma band 180 is similar to the stoma band 100, but includes the storage pouch 186. The storage pouch 186 may be used to store extra collection devices, cleaning wipes, or other items as desired. The storage pouch 186 can include a sealing means 188 that can be, for example, a zipper, an interlocking press-fit seal, hook and loop fasteners, buttons, snaps, and the like. The storage pouch 186 may be located along a length of the belt portion 182 as desired. The storage pouch may also be located on an inside of the belt portion 182 or an outside of the belt portion 182 as desired.

Referring now to FIG. 6, an isometric view of a stoma band 200 that includes supportive straps is shown. The stoma band 200 includes a belt portion 202 with a contoured portion 204, fasteners 206, shoulder straps 208, and leg straps 210. The stoma band 200 is similar to the stoma band 100, but includes the shoulder straps 208 and the leg straps 210. Depending on the shape of the user's body, the stoma band 200 may not stay in place to cover the user's stoma. In order to help maintain the desired placement of the stoma band 200 around the user's body, the shoulder straps 208 may be used to help prevent the belt portion 202 from sliding down the user's body. Similarly, the leg straps 210 may be used to help prevent the belt portion 202 from sliding up the user's body. In various embodiments, the stoma band 200 may include both the shoulder straps 208 and the leg straps 210, only the shoulder straps 208, or only the leg straps 210. As shown in FIG. 6, the fasteners 206 include multiple sets of hook and loop fasteners. In various embodiments, the fasteners 206 may instead include one set of hook and loop fasteners, similar to the embodiments of FIGS. 1-5. In lieu of straps, placement of the stoma band 200 can be maintained in other ways. For example, the stoma band 200 may be secured to a user's garments via, for example, buttons, hook and look fasteners, and the like. In some embodiments, the stoma band 200 may attach to a user's pants, shorts, underwear, etc. In some embodiments, the stoma band 200 may attach to a user's shirt or the like. In some embodiments, the stoma band 200 may be integrated into an article of clothing, such as, for example, a form fitting shirt or undergarment (e.g., compression shirts, shorts, and the like). In such an embodiment, the stoma band 200 and the article of clothing are a unitary garment.

Referring now to FIG. 7, an illustration of a stoma band system 220 secured around a stoma 226 of a user 10 is shown. The stoma band system 220 includes a belt portion 222 and a collection device 228. The belt portion 222 may include similar features to those included in the embodiments of FIGS. 1-6. The belt portion 222 includes a fastener 224 that is similar to the fasteners discussed above. The belt portion 222 is positioned on the user 10 so that the belt portion 222 covers the stoma 226 and an area of skin surrounding the stoma 226. The collection device 228 is positioned between the belt portion 222 and the user 10, and proximal to the stoma 226 to generally cover the stoma 226. In some embodiments, the collection device 228 is removably secured to the belt portion 222 by hook and look fasteners, buttons, snaps, and the like. In some embodiments, the collection device 228 is not directly affixed to the belt portion 222, but is constrained by its placement between a user's body and the belt portion 222.

A size of the collection device 228 may be varied as desired. For example, in some embodiments, smaller collection devices 228 that generally only cover the stoma 226 may be used. In other embodiments, larger collection devices 228 may be used that extend across a user's entire waste. Larger collection devices 228 tend to have a greater absorptive capacity, and can extend an amount of time that the user can use the collection device 228 before it is necessary to insert a new collection device 228. With the collection device 228 positioned over the stoma 226, the collection device 228 is able collect any waste that is evacuated out of the stoma 226.

In the embodiment shown in FIG. 7, the stoma band system 220 is adapted for urostomy and the collection device 228 is an absorptive pad that can absorb, for example, urine. Different types of absorptive materials may be used, such as, for example, bamboo, cotton, hemp, modal fabrics, polyester, fleece, wool, PUL (Polyurethane Laminate, e.g., a blend of 83% polyester, 15% polyurethane, and 2% agglutinant), and the like. In some embodiments, the collection device 228 includes a backing material that blocks leakage of absorbed waste from seeping through a back side of the collection device.

Although only one stoma 226 is shown in FIG. 7, additional stomas 226 may be present. If, for example, a second stoma 226 is present, a second collection device 228 may be positioned to collect waste evacuated from the second stoma 226, or a collection device that is large enough to cover both stomas may be used. In some embodiments, medication may be applied to the collection device 228 so that the collection device 228 serves as both an absorption mechanism and as a device capable of providing medical treatment or therapy to the user's skin. Various medications may be applied to the collection device 228, such as, for example, lotions, creams, ointments, salves, and the like.

During use of the stoma band system 220, it will become necessary to remove a collection device 228 as it becomes saturated with waste. To replace a used collection device 228, the user can undo the fastener 224 to open the belt portion 222. The used collection device can then be removed and replaced with a new collection device 228. This process can be repeated as needed to make sure that the collection device 228 in use does not become over saturated to the point of leaking.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Although various embodiments of the method and system of the present invention have been illustrated in the accompanying Drawings and described in the foregoing Specification, it will be understood that the invention is not limited to the embodiments disclosed, but is capable of numerous rearrangements, modifications, and substitutions without departing from the spirit and scope of the invention as set forth herein. For example, and not by way of limitation, features such as the contoured portions, storage pouch, shoulder straps, and leg straps may be added to or removed from the various embodiments discussed herein as desired. It is intended that the Specification and examples be considered as illustrative only.

1. A stoma band comprising:
a belt portion having a first side that faces away from a user wearing the stoma band, a second side that faces toward the user wearing the stoma band, a first end, and a second end;
a first fastener on the first end of the belt portion and a second fastener on the second end of the belt portion; and
a collection device adapted to be positioned between the second side of the belt portion and the user so that the collection device is proximal to a stoma of the user.

2. The stoma band of claim 1, further comprising a second collection device adapted to be positioned between the second side of the belt portion and the user so that the second collection device is proximal to a second stoma of the user.

3. The stoma band of claim 1, wherein the collection device is removably secured to the second side of the belt portion by at least one of a hook and loop fastener, a button, and a snap.

4. The stoma band of claim 1, wherein the collection device is made of a material comprising at least one of bamboo, cotton, hemp, modal fabrics, polyester, fleece, wool, and polyurethane laminate.

5. The stoma band of claim 1, wherein the first fastener comprises at least one removable fastener that can be removed to adjust a length of the belt portion.

6. The stoma band of claim 5, further comprising a cut-line disposed between adjacent removable fasteners.

7. The stoma band of claim 1, further comprising a contoured portion disposed along a length of the belt portion.

8. The stoma band of claim 6, further comprising a second contoured portion disposed along the length of the belt portion.

9. The stoma band of claim 1, wherein the second end comprises a tapered end.

10. The stoma band of claim 1, wherein the first fastener is disposed on the first side of the belt portion and the second fastener is disposed on a second side of the belt portion.

11. The stoma band of claim 1, further comprising a shoulder strap attached to the belt portion.

12. The stoma band of claim 1, further comprising a leg strap attached to the belt portion.

13. The stoma band of claim 1, wherein the stoma band is attached to at least one of a shirt, pants, shorts, and an undergarment.

14. The stoma band of claim 1, wherein the stoma band is integrated into an article of clothing.

15. The stoma band of claim 1, wherein the first and second fasteners are hook and loop fasteners.

16. The stoma band of claim 1, wherein the first and second fasteners are button snaps.

17. The stoma band of claim 1, wherein the first and second fasteners are buckles.

18. The stoma band of claim 1, further comprising a pouch disposed on the belt portion.

19. The stoma band of claim 18, wherein the pouch is disposed on the second side of the belt portion.

20. The stoma band of claim 1, wherein the collection device extends across a waste of the user.
